SEQUENCE LISTING (7 sequences)

SEQ ID NO: 1 - DNA, 38 bp, Artificial Sequence
  Synthetic
aaaaaaaaaactgttttcctttacttactacacctcag

SEQ ID NO: 2 - DNA, 53 bp, Artificial Sequence
  Synthetic
aaaaaaaaaagaccttcaatgactttctagtaactcagcagcatctcagggcc

SEQ ID NO: 3 - DNA, 42 bp, Artificial Sequence
  Synthetic
aaaaaaaaaagaaaactgtaacaataagagtggagatagctg

SEQ ID NO: 4 - DNA, 40 bp, Artificial Sequence
  Synthetic
aaaaaaaaaatcaaagaatggtcctgcaccagtaatatgc

SEQ ID NO: 5 - DNA, 35 bp, Artificial Sequence
  Synthetic
aaaaaaaaaaggtgaagcagaatcagtggtcgccc

SEQ ID NO: 6 - DNA, 40 bp, Artificial Sequence
  Synthetic
aaaaaaaaaaaggattcccagagcccacaatagattggta

SEQ ID NO: 7 - DNA, 42 bp, Artificial Sequence
  Synthetic
aaaaaaaaaataatcatctcacctctgctcagttcctggaca

(19) United States
(12) Patent Application Publication
(10) Pub. No.: US 2014/0287408 A1
(43) Pub. Date: Sep. 25, 2014

(54) TARGET SEQUENCE ENRICHMENT

(71) Applicant: Abbott Molecular Inc., Des Plaines, IL (US)

(72) Inventors: Hong Su, Evanston, IL (US); Shihai Huang, Lincolnshire, IL (US)

(73) Assignee: Abbott Molecular Inc., Des Plaines, IL (US)

(21) Appl. No.: 14/209,629

(22) Filed: Mar. 13, 2014

Related U.S. Application Data
(60) Provisional application No. 61/780,204, filed on Mar. 13, 2013.

(51) Int. Cl. C12Q 1/68

(52) U.S. Cl. CPC C12Q 1/6806
USPC 435/6.11

(57) ABSTRACT

The present invention provides methods, systems, kits, and compositions for magnetically purifying target nucleic acid sequences from a sample using bait molecules configured to bind both target nucleic acid sequences and magnetic binding particles. In certain embodiments, the bait molecules comprise a short target capture sequence (e.g., 18 to 48 bases), and the methods employ a short hybridization time (e.g., 1-4 hours) and a low hybridization temperature (e.g., about room temperature).

The present application claimed priority to U.S. Provisional application 61/780,204, filed Mar. 13, 2013, which is herein incorporated by reference in its entirety.

### FIELD OF THE INVENTION

The present invention provides methods, systems, kits, and compositions for magnetically purifying target nucleic acid sequences from a sample using bait molecules configured to bind both target nucleic acid sequences and magnetic binding particles. In certain embodiments, the bait molecules comprise a short target capture sequence (e.g., 18 to 39 bases), and the methods employ a short hybridization time (e.g., 1-4 hours) and a low hybridization temperature (e.g., about room temperature).

### BACKGROUND

Detection of mutations across large genomic regions is becoming increasingly important for clinical diagnosis and pharmacogenetics. Common techniques for mutation detection include real-time PCR and Sanger Sequencing, but these technologies have their limitations, such as limited multiplex capability for real-time PCR, and mutation detection sensitivity for Sanger sequencing. Next-Gen sequencing has great multiplex capability and can generate several gigabases of sequences in one run, thus enabling its potential applicability in clinical applications on mutation detections. However, due to the size of human genome and heterogeneity of tumors, whole genome sequencing is not suitable for highly sensitive mutation detection and remains prohibitively expensive for wide adoption in clinical diagnostics. Current clinical applications are mainly focused on highly sensitive detection of specific known biomarkers and high-risk factors rather than complete genomic sequencing.

To enable these clinical applications, an enrichment step can be performed prior to sequencing amplification to increase the amount of targeted sequences relative to non targeted sequences, therein to increase coverage of genes of interest with a given sequencing load and to further reduce the required amount of sequencing load. Target enrichment generally refers to a methodology where genomic regions of interest are selectively made to be over-represented from a DNA sample before sequencing. Three general target-enrichment strategies have been described: PCR, Molecular inversion probe (MIP) (Nilsson et al., Science 1994, 265: 2085-2088; Hardenbol et al., Nat Biotechnol 2003, 21: 673-678; and Porreca et al., Nat methods 2007, 4, 931-936; all of which are herein incorporated by reference), and Hybridization-based capture (Okou et al. Nat Methods 2007, 4: 907-909; and Albert et al. Nat Methods 2007, 4: 903-905; both of which are herein incorporated by reference).

PCR is the most often used method for target enrichment. PCR methods are specific and efficient. However, their general lack of multiplex capability makes this enrichment strategy cumbersome and costly. Molecular inversion probe (MIP) is composed of two consecutive target-specific sequences separated by a linker. The probe forms a circle like a padlock when hybridized to the target DNA, thereby the target region may be amplified by PCR using the common linker sequences. Hybridization-based capture utilizes baits that are designed to hybridize to selected target regions from the DNA pool. The hybridization-based capture methodologies come in two formats: solid surface based methods (such as micro-array) and solution based methods (such as SURESELECT XT target enrichment kit by Agilent, SEQCAP EZ by Roche-NimbleGen, TRUSEQ EXOME capture Product by Illumina; and RIVIA Target Enrichment by Rivia). Hybridization based capture can be used to generate libraries both for multiple target loci and for multiple samples (each with identifiable index sequences) in one reaction.

### SUMMARY OF THE INVENTION

The present invention provides methods, systems, kits, and compositions for magnetically purifying target nucleic acid sequences from a sample using bait molecules configured to bind both target nucleic acid sequences and magnetic binding particles (e.g., paramagnetic binding particles). In certain embodiments, the bait molecules comprise a short target capture sequence (e.g., 18 to 39 bases), and the methods employ a short hybridization time (e.g., 1-4 hours) and a low hybridization temperature (e.g., about room temperature). In certain embodiments, hybridization is conducted at a high salt concentration, such as at least 1.3 M (e.g., 1.5-2.5 M).

In some embodiments, the present invention provides methods of separating target nucleic acid sequences from a target sample comprising: a) contacting a target sample with bait molecules to generate a mixed sample, wherein the target sample comprises a population of target and non-target nucleic acid sequences (e.g., greater than 95% . . . 99% . . . or 99.999% are non-target nucleic acid sequences), and wherein the bait molecules: i) are free in solution in the mixed sample, and ii) each comprises a ligand (e.g., biotin) and a target capture sequence (e.g., each bait molecule has the same target capture sequence or one of many different target capture sequences for multiplex purification) which is 15 to 49 bases in length (e.g., 15 . . . 20 . . . 25 . . . 30 . . . 35 . . . 40 . . . 45 . . . 49 bases in length); b) heating the mixed sample to a nucleic acid denaturation temperature (e.g., 80-99 degrees Celsius); c) incubating the mixed sample (e.g., with a salt concentration of at least 1.3M or at least 1.9M) at a hybridization temperature such that the target capture sequences hybridize to the target nucleic acid sequences, wherein the incubating is conducted for no more than 18 hours (e.g., no more than 18 . . . 15 . . . 12 . . . 8 . . . 5 . . . 4.5 . . . 4.0 . . . 3.0 . . . 2.2 . . . 1.9 . . . 1.5 . . . 1.1 . . . or 0.8 hours) before performing steps d), e) and f); d) adding magnetic binding particles (e.g., paramagnetic binding particles) to the mixed sample, wherein the magnetic binding particles comprise ligand binding moieties (e.g., streptavidin); e) incubating the mixed sample under conditions such that the bait molecules bind to the magnetic binding particles via the ligands binding to the ligand binding moieties thereby generating a population of target nucleic acid sequence linked magnetic particles; and f) magnetically separating the target nucleic acid sequence linked magnetic binding particles from the mixed sample thereby generating a population of separated target nucleic acid sequence linked magnetic binding particles.

In some embodiments, the mixed sample, during said incubating in step c), has, or is treated to have, a salt concentration of at least 1.1 M (e.g., 1.1 . . . 1.3 . . . 1.5 . . . 1.7 . . . 1.9 . . . 2.0 . . . 2.1 . . . 2.7 or about 2 M). The present invention is noted limited how the mixed sample is treated to achieve this salt concentration. A salt and/chaotropic agent can be added to the mixed sample to achieve the salt concentration. Salts and chaotropic agents for inclusion in samples include, but are not limited to: trichloroacetate, thiocyanate, guanidinium salts, butanol, ethanol, guanidinium chloride, lithium perchlorate, lithium acetate, magnesium chloride, phenol, propanol, sodium dodecyl sulfate, thiourea, urea, and guanidinium thiocyanate. In certain embodiments, prior to hybridization, the mixed sample is treated with a hybridization buffer that contains a chaotropic agent. In certain embodiments, the chaotropic agent is guanidine cyanate. In other embodiments, the buffer contains Tris or other buffer agent.

In certain embodiments, the incubating in step c) is conducted for no more than 1.5 hours before performing steps d), e) and f). In certain embodiments, the target capture sequence is 22-35 bases in length. In further embodiments, the target capture sequence is 25-32 bases in length. In other embodiments, the hybridization temperature in step c) is about 15-30 degrees Celsius (e.g., about 15 . . . 18 . . . 21 . . . 24 . . . 27 or 30 degrees Celsius). In other embodiments, the hybridization temperature in step c) is about room temperature (e.g. about 21, 22, 23, 24, 25, or 26 degrees Celsius). In further embodiments, the methods further comprise washing the population of separated target nucleic acid sequence linked magnetic particles with a wash solution. In particular embodiments, the washing is conducted at a temperature of about 30-50 degrees Celsius (e.g., about 30 . . . 35 . . . 40 . . . 45 . . . or 50 degrees Celsius).

In some embodiments, in step a), the target sample and the bait molecules are further contacted with carrier nucleic acid (e.g. to block non-specific binding) in order to generate the mixed sample. In particular embodiments, the carrier nucleic acid comprises blocking oligonucleotides and/or human repetitive nucleic acid sequences (e.g., Cot-1 sequences or Alu sequences). In further embodiments, the methods further comprise contacting the population of separated target nucleic acid sequence linked magnetic binding particles with an aqueous solution (e.g., at a denaturation temperature), and eluting the target nucleic acid sequences away from the magnetic binding particles to generate a population of eluted target nucleic acid sequences. In certain embodiments, the population of eluted target nucleic acid sequences are subjected to sequencing (e.g., next generation sequencing techniques). In other embodiments, the population of eluted targeted nucleic acid sequences are subjected to amplification (e.g., PCR, whole genome amplification, etc.). In other embodiments, the total amount of nucleic acid in the target sample is between 100 nanograms and 5.0 micrograms (e.g., 100 nanograms . . . 500 nanograms . . . 1.0 microgram . . . 3.5 micrograms . . . 5.0 micrograms). In further embodiments, the total amount of nucleic acid in the target sample is between 200 nanograms and 2.0 micrograms.

In further embodiments, the target sample comprises a total nucleic acid preparation from lysed human cells. In other embodiments, the target nucleic acid sequences are human nucleic acid sequences. In further embodiments, the target nucleic acid sequences are pathogenic nucleic acid sequences (e.g., virus, bacteria, fungi, etc.) and the non-target nucleic acid sequences are human nucleic acid sequences. In some embodiments, the ligand is selected from the group consisting of: biotin, streptavidin, an antibody specific for the ligand binding moiety, and a protein bound by the ligand binding moiety. In other embodiments, the ligand binding moieties are selected from the group consisting of: biotin, streptavidin, an antibody specific for the ligand, and a protein bound by the ligand. In additional embodiments, the bait molecules further comprise a linker moiety, wherein the linker moiety is located between the ligand and the target capture sequence. In certain embodiments, the linker moiety is selected from the group consisting of: tetra-ethyleneglycol and a carbon chain linker 2-40 carbon atoms in length.

In certain embodiments, the denaturation temperature is about 80-100 degrees Celsius (e.g., about 80 . . . 85 . . . 90 . . . 95 . . . or 100 degrees Celsius). In further embodiments, the target nucleic acid sequences are DNA or RNA. In other embodiments, the magnetic binding particles comprise iron and are in the shape of beads.

In some embodiments, the magnetically separating comprises: i) inserting a tube containing the target nucleic acid sequence linked magnetic binding particles (e.g., paramagnetic binding particles) into a magnetic rack such that the target nucleic acid sequence linked magnetic binding particles are captured on the sides of the tube; and ii) removing all or nearly all of the contents of the tube that is not captured on the walls of the tube. In further embodiments, the method further comprises removing the tube from the magnetic rack and adding an aqueous solution to the tube. In certain embodiments, the target nucleic acid sequences comprise at least first and second different types of target nucleic acid sequences, and some of the bait molecules comprise a target capture sequence specific for the first different type of target nucleic acid sequence, and some of the bait molecules comprise a target capture sequence specific for the second different type of target nucleic acid sequence.

In particular embodiments, the present invention provides methods of separating target nucleic acid sequences from a target sample comprising: a) contacting a target sample with bait molecules to generate a mixed sample, wherein the target sample comprises a population of target and non-target nucleic acid sequences, and wherein the bait molecules: i) are free in solution in the mixed sample, and ii) each comprises a ligand (e.g., biotin) and a target capture sequence which is 20 to 44 bases in length; b) heating the mixed sample to a nucleic acid denaturation temperature; c) incubating the mixed sample at about room temperature such that the target capture sequences hybridize to the target nucleic acid sequences, wherein the incubating is conducted for no more than 2 hours before performing steps d), e) and f); d) adding magnetic binding particles (e.g., paramagnetic binding particles) to the mixed sample, wherein the magnetic binding particles comprise ligand binding moieties (e.g., streptavidin molecules); e) incubating the mixed sample under conditions such that the bait molecules bind to the magnetic binding particles via the ligands binding to the ligand binding moieties thereby generating a population of target nucleic acid sequence linked magnetic particles; and f) magnetically separating the target nucleic acid sequence linked magnetic binding particles from the mixed sample thereby generating a population of separated target nucleic acid sequence linked magnetic binding particles.

In further embodiments, the present invention provides compositions or systems comprising: a) a solution comprising bait molecules, wherein the bait molecules are free in solution, and wherein the bait molecules comprise a ligand and a target capture sequence 18 to 48 (e.g., 18 . . . 24 . . . 29 . . . 35 . . . 41 . . . or 48) bases in length; b) magnetic binding particles (e.g., paramagnetic binding particles), wherein the magnetic binding particles comprise ligand binding moieties. In additional embodiments, the target capture sequence is 25-33 bases in length. In other embodiments, the ligand comprises biotin and the ligand binding moieties comprise streptavidin. In certain embodiments, the solution has a salt concentration of at least 1.1 M (e.g., at least 1.1 . . . 1.3 . . . 1.7 . . . 1.9 . . . 2.0 . . . 2.7 M; about 2M; or about 1.5 to 2.5 M).

In some embodiments, the present invention provides methods of separating target nucleic acid sequences from a target sample comprising: a) contacting a target sample with bait molecules and carrier DNA to generate a mixed sample, wherein the target sample comprises a population of target and non-target nucleic acid sequences, and wherein the bait molecules: i) are free in solution in the mixed sample, and ii) each comprises a ligand (e.g., biotin) and a target capture sequence which is 25 to 33 bases in length; b) heating the mixed sample to a nucleic acid denaturation temperature (e.g., about 92 degrees Celsius); c) incubating the mixed sample at about room temperature such that the target capture sequences hybridize to the target nucleic acid sequences, wherein the incubating is conducted for no more than about 1 hour before performing steps d), e) and f); d) adding magnetic binding particles (e.g., paramagnetic binding particles) to the mixed sample, wherein the magnetic binding particles comprise ligand binding moieties (e.g., streptavidin molecules); e) incubating the mixed sample under conditions (e.g., at about room temperature for about 5-20 minutes) such that the bait molecules bind to the magnetic binding particles via the ligands binding to the ligand binding moieties (e.g., biotin binding to streptavidin) thereby generating a population of target nucleic acid sequence linked magnetic particles; and f) magnetically separating the target nucleic acid sequence linked magnetic binding particles from the mixed sample thereby generating a population of separated target nucleic acid sequence linked magnetic binding particles.

In particular embodiments, the present invention provides methods of separating target nucleic acid sequences from a target sample comprising: a) contacting a target sample with bait molecules and carrier DNA to generate a mixed sample, wherein the target sample comprises a population of target and non-target nucleic acid sequences, and wherein the bait molecules: i) are free in solution in the mixed sample, and ii) each comprises a ligand (e.g., biotin) and a target capture sequence which is 25 to 32 bases in length; b) heating the mixed sample to a nucleic acid denaturation temperature (e.g., about 92 degrees Celsius); c) incubating the mixed sample at about room temperature such that the target capture sequences hybridize to the target nucleic acid sequences, wherein the incubating is conducted for no more than about 1 hour before performing steps d), e) and f); d) adding magnetic binding particles to the mixed sample, wherein the magnetic binding particles comprise ligand binding moieties (e.g., streptavidin molecules); e) incubating the mixed sample under conditions (e.g., at about room temperature for about 5-20 minutes) such that the bait molecules bind to the magnetic binding particles via the ligands binding to the ligand binding moieties (e.g., biotin binding to streptavidin) thereby generating a population of target nucleic acid sequence linked magnetic particles; f) magnetically separating the target nucleic acid sequence linked magnetic binding particles from the mixed sample thereby generating a population of separated target nucleic acid sequence linked magnetic binding particles; g) removing all or nearly all of the liquid from the separated target nucleic acid sequence linked magnetic binding molecules; and h) adding a buffer to said separated target nucleic acid sequence linked magnetic binding molecules, and removing magnetization, in order to generate a suspension.

In some embodiments, the present invention provides methods where the target capture sequences are directly linked to the magnetic binding particles. For example, in some embodiments, the present invention provides methods of separating target nucleic acid sequences from a target sample comprising: a) contacting a target sample with magnetic binding molecules linked to target capture sequences that are 18 to 39 bases in length to generate a mixed sample, wherein said target sample comprises a population of target and non-target nucleic acid sequences; b) heating said mixed sample to a nucleic acid denaturation temperature; c) incubating said mixed sample at a hybridization temperature such that said target capture sequences hybridize to said target nucleic acid sequences thereby generating target nucleic acid sequence linked magnetic binding particles, wherein said incubating is conducted for no more than 4 hours before performing step d); and d) magnetically separating said target nucleic acid sequence linked magnetic binding particles from said mixed sample thereby generating a population of separated target nucleic acid sequence linked magnetic binding particles.

### DESCRIPTION OF THE FIGURE

FIG. 1 shows an exemplary flow chart of one embodiment of the target purification methods of the present invention.

### DEFINITIONS

As used herein, the term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method.

As used herein, the term “sample” refers to anything capable of being analyzed by the methods provided herein that is suspected of containing a target nucleic acid sequence. Samples may be complex samples or mixed samples, which contain nucleic acids comprising multiple different nucleic acid sequences. Samples may comprise nucleic acids from more than one source (e.g. difference species, different subspecies, etc.), subject, and/or individual. In some embodiments, the methods provided herein comprise purifying the sample or purifying the nucleic acid(s) from the sample. In some embodiments, the sample contains purified nucleic acid. In some embodiments, a sample is derived from a biological, clinical, environmental, research, forensic, or other source.

As used herein, the phrase “bait molecules” refers to molecules configured to bind both target nucleic acid sequences and magnetic binding particles. In particular embodiments, the bait molecules comprise a ligand and a target capture sequence.

As used herein, the term “ligand” refers to any type of moiety that is capable of being bound by a ligand binding moiety. Examples of ligands include, but are not limited to, biotin, streptavidin, antibodies, etc.

### DETAILED DESCRIPTION

The present invention provides methods, systems, kits, and compositions for magnetically purifying target nucleic acid sequences from a sample using bait molecules configured to bind both target nucleic acid sequences and magnetic binding particles. In certain embodiments, the bait molecules comprise a short target capture sequence (e.g., 18 to 39 bases), and the methods employ a short hybridization time (e.g., 1-4 hours) and a low hybridization temperature (e.g., about room temperature).

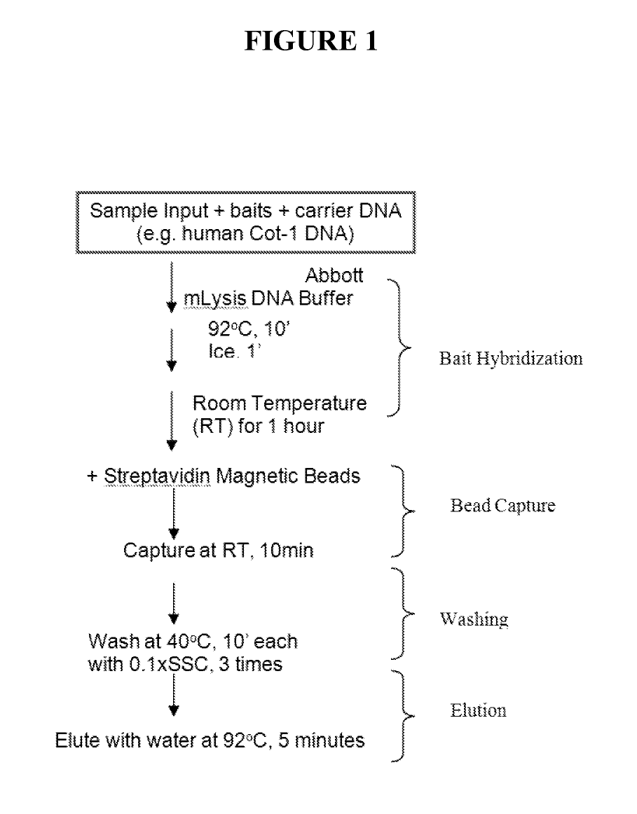

FIG. 1 shows an exemplary embodiment of the present invention. As shown in this FIGURE, sample input (suspected of containing target nucleic acid sequences) are combined with baits (e.g., biotinylated baits containing sequences complementary to target sequences) and carrier DNA (e.g., Cot-1 DNA). To this mixture, mLysis DNA buffer (Abbott) is added in a final concentration of 45% and the mixture is heated to 92 degrees Celsius for about 10 minutes. The mixture is then chilled on ice for 1 minute and then incubated at room temperature for 1 hour to allow the sequences on the baits to hybridize to target sequences from the sample. Streptavidin coated magnetic beads are then added to the mixture, which is incubated for 10 minutes such that the hybridized sequences are captured by the streptavidin-coated magnetic beads. The mixture is then moved into a magnetic rack to allow binding of the beads to the side of the container and removal of the remaining liquid in the container, then moving the mixture out of the magnetic rack, follow by re-suspension of the beads in buffer. Moving the mixture in and out of the magnetic racks can be repeated a number of times to allow washing of the particles with a wash solution and complete separation of the magnetic particles (with bound target sequences) from the original sample. As shown in FIG. 1, the washing steps can be conducted at 40 degrees Celsius, for 10 minutes, using 0.1×SSC, and can be conducted 3 times. Finally, the captured target nucleic acid sequences can be eluted off the beads using water and a temperature of 92 degrees Celsius for 5 minutes (thereby generating a final aqueous preparation containing purified target nucleic acid sequences). The purified target nucleic acid sequences can be used in further methods, as described below.

While the present invention is not limited to any particular mechanism or theory of operation, it is believed that it has advantages over the prior art as follows. For example, in certain embodiments, the target binding sequences are relatively short (e.g., 25-43 bases in length), which is believed to be shorter than current commercial hybridization target enrichment products. Shorter baits allow for faster hybridization kinetics and lower hybridization temperature without compromising hybridization specificity, while longer baits have better hybridization stability and may tolerate mismatches and deletion better than shorter baits. In certain embodiments, blocking reagents are employed and can be important to prevent non-specific hybridization. Human Cot1 DNA or other carrier DNAs containing human sequences can be used to prevent non-targeted DNA molecules from being pulled down along with target molecules due to non-specific hybridization. By similar theory, blocking oligonucleotides can also be used if the DNA sample is, for example, end-ligated with universal adapters. The reason is that adaptors have the same sequence, and therefore, similar to repetitive sequences in the genome, adapter sequences may hybridize to each other during enrichment, and thereby be captured as non-specific fragments.

In certain embodiments, the methods of the present invention allow a purified preparation of target sequences to be generated in less than 4 hours (or less than 3 or 2 hours) starting from an initial target sample. In particular embodiments, the method is conducted in an automated or partially automated manner, using machines such as the m2000sp (Abbott) or similar systems.

In certain embodiments, the carrier DNA is human Cot-1 DNA (e.g., from Invitrogen). In particular embodiments, the magnetic beads are NANOLINK streptavidin magnetic beads from SOLULINK. In further embodiments, the hybridization buffer employed is saline-sodium citrate (SSC) buffer (e.g., from Promega). In certain embodiments, the magnetic beads are selected from the following: Sera-Mag* Magnetic Streptavidin Particles (SeraDyne); Dynabeads® M-280 Streptavidin (Invitrogen); Dynabeads® M-270 Streptavidin (Invitrogen); Dynabeads® MyOne™ Streptavidin C1 (Invitrogen); Dynabeads® MyOne™ Streptavidin T1 (Invitrogen) and Promega streptavidin MagneSphere.

While the present invention is not limited to any particular mechanism or theory, it is believed that the solution based capture methods of the present invention have certain advantages over the prior art (e.g., advantages over PCR, MIP, and solid surface based hybridization). For example, the solution based hybridization methods of the present invention requires far less DNA materials compared with solid surface based methods because, for example, high concentration of baits can be used to drive the hybridization thermodynamics and kinetics, thereby resulting in a more efficient enrichment. As such, in certain embodiments, sample input is only 250 ng to 1 microgram genomic DNA. In certain embodiments, the methods of the present invention utilize baits with short capture sequences (e.g., 25-32 bases or 20-39 bases in length). The use of short capture sequences allow for low hybridization temperature (e.g. room temperature) and washing temperature (e.g. 40 degree) to maximize binding efficiency without sacrificing specificity significantly. Short capture sequences also support fast hybridization kinetics. Furthermore, the manufacturing of the short capture sequences can use standard synthesis procedures (e.g., for biotinylated oligonucleotides). Such manufacturing procedures are well established, efficient and cost efficient. Another advantage, in certain embodiments, is that the reaction involved with the hybridization between targets and baits and coupling of target/bait complexes to the beads are non-enzymatic processes largely independent of specific sequence context. High level of multiplex reaction (i.e. number of different targeted sequences) can be achieved without additional technical requirements compared with single target reactions. This is unlike PCR, where technical limitation in high level multiplex reactions is still significant.

In some embodiments, a sample is analyzed for the presence and/or abundance of a target nucleic acid sequences in a potentially complex sample which may contain many different nucleic acid sequences, each of which may or may not contain the target sequence. In some embodiments, a sample is analyzed to determine the proportion of nucleic acid molecules containing a target sequence of interest. In some embodiments, a complex sample is analyzed to detect the presence and/or measure the abundance or relative abundance of multiple target sequences (e.g., multiple different capture sequences are employed). In some embodiments, methods provided herein are used to determine what sequences are present in a mixed sample and/or in what relative proportions.

In certain embodiments, the purified target nucleic acid sequences are generated by the methods of the present invention are subjected to amplification. Exemplary amplification reactions include, but are not limited to the polymerase chain reaction (PCR) or ligase chain reaction (LCR), each of which is driven by thermal cycling. Amplifications used in method or assays of the present invention may be performed in bulk and/or partitioned volumes (e.g. droplets). Alternative amplification reactions, which may be performed isothermally, also find use herein, such as branched-probe DNA assays, cascade-RCA, helicase-dependent amplification, loop-mediated isothermal amplification (LAMP), nucleic acid based amplification (NASBA), nicking enzyme amplification reaction (NEAR), PAN-AC, Q-beta replicase amplification, rolling circle replication (RCA), self-sustaining sequence replication, strand-displacement amplification, and the like.

Amplification may be performed with any suitable reagents (e.g. template nucleic acid (e.g. DNA or RNA), primers, probes, buffers, replication catalyzing enzyme (e.g. DNA polymerase, RNA polymerase), nucleotides, salts (e.g. MgCl₂), etc. In some embodiments, an amplification mixture includes any combination of at least one primer or primer pair, at least one probe, at least one replication enzyme (e.g., at least one polymerase, such as at least one DNA and/or RNA polymerase), and deoxynucleotide (and/or nucleotide) triphosphates (dNTPs and/or NTPs), etc.

In some embodiments, the present invention utilizes nucleic acid amplification that relies on alternating cycles of heating and cooling (i.e., thermal cycling) to achieve successive rounds of replication (e.g., PCR). In some embodiments, PCR is used to amplify target nucleic acids (e.g. partitioned targets). PCR may be performed by thermal cycling between two or more temperature set points, such as a higher melting (denaturation) temperature and a lower annealing/extension temperature, or among three or more temperature set points, such as a higher melting temperature, a lower annealing temperature, and an intermediate extension temperature, among others. PCR may be performed with a thermostable polymerase, such as Taq DNA polymerase (e.g., wild-type enzyme, a Stoffel fragment, FastStart polymerase, etc.), Pfu DNA polymerase, S-Tbr polymerase, Tth polymerase, Vent polymerase, or a combination thereof, among others. Typical PCR methods produce an exponential increase in the amount of a product amplicon over successive cycles, although linear PCR methods also find use in the present invention.

Any suitable PCR methodology, combination of PCR methodologies, or combination of amplification techniques may be utilized for amplification and detection, such as allele-specific PCR, assembly PCR, asymmetric PCR, digital PCR, endpoint PCR, hot-start PCR, in situ PCR, intersequence-specific PCR, inverse PCR, linear after exponential PCR, ligation-mediated PCR, methylation-specific PCR, miniprimer PCR, multiplex ligation-dependent probe amplification, multiplex PCR, nested PCR, overlap-extension PCR, polymerase cycling assembly, qualitative PCR, quantitative PCR, real-time PCR, RT-PCR, single-cell PCR, solid-phase PCR, thermal asymmetric interlaced PCR, touchdown PCR, or universal fast walking PCR, etc.

In some embodiments, qualitative PCR is used to detect target nucleic acid sequences in a purified sample obtained by the methods described herein. In some embodiments, qualitative PCR-based analysis determines whether or not a target is present in a sample, generally without any substantial quantification of target. In some embodiments, digital PCR that is qualitative may be performed by determining whether a partition or droplet is positive for the presence of target. In some embodiments, qualitative digital PCR is used to determine the percentage of partitions that are positive for the presence of target. In some embodiments, qualitative digital PCR is used to determine whether a particular number of droplets contains at least a threshold percentage of positive droplets (i.e. a positive sample). In some embodiments, qualitative PCR is performed to detect the presence of multiple targets in a sample.

In some embodiments, a purified target preparation is assayed to determine the presence of the target sequence (or amplicons thereof) or specific SNPs or stretches of bases therein. In some embodiments, the present invention provides systems, devices, methods, and compositions to identify the presence of nucleic acids (e.g. amplicons, labeled nucleic acids) in a purified target sequence sample. In some embodiments, fluorescence detection methods are provided for detection of target nucleic acid. For example, the protocols may employ reagents suitable for use in a TaqMan reaction, such as a TaqMan probe; reagents suitable for use in a SYBR Green fluorescence detection; reagents suitable for use in a molecular beacon reaction, such as molecular beacon probes; reagents suitable for use in a scorpion reaction, such as a scorpion probe; reagents suitable for use in a fluorescent DNA-binding dye-type reaction, such as a fluorescent probe; and/or reagents for use in a LightUp protocol, such as a LightUp probe. In some embodiments, the present invention provides methods and compositions for detecting and/or quantifying a detectable signal (e.g. fluorescence) from partitions containing amplified target nucleic acid. Thus, for example, methods may employ labeling (e.g. during amplification, post-amplification) amplified nucleic acids with a detectable label, exposing partitions to a light source at a wavelength selected to cause the detectable to fluoresce, and detecting and/or measuring the resulting fluorescence. Fluorescence emitted from the partitions can be tracked during amplification reaction to permit monitoring of the reaction (e.g., using a SYBR Green-type compound), or fluorescence can be measure post-amplification.

In some embodiments, the present invention provides methods of detecting and/or quantifying the presence of a target nucleic acid in a purified preparation by providing a probe with specificity for a target nucleic acid (e.g., a TaqMan-type probe), and detecting the resulting fluorescence. In some embodiments, samples containing amplified target nucleic acid will exhibit post-amplification fluorescence. In some embodiments, detection of a fluorescent signal is indicative of the presence of the target nucleic acid (e.g. amplified target) in the sample.

The present invention provides corresponding methods for using other suitable target-specific probes (e.g. intercalation dyes, scorpion probes, molecular beacons, etc.), as would be understood by one of skill in the art. In some embodiments, the present invention provides detection of samples containing amplified nucleic acids and/or the amplicons contained therein, using one or more of fluorescent labeling, fluorescent intercalation dyes, FRET-based detection methods (U.S. Pat. No. 5,945,283; PCT Publication WO 97/22719; both of which are incorporated by reference in their entireties), quantitative PCR, real-time fluorogenic methods (U.S. Pat. No. 5,210,015 to Gelfand, U.S. Pat. No. 5,538,848 to Livak, et al., and U.S. Pat. No. 5,863,736 to Haaland, as well as Heid, C. A., et al., Genome Research, 6:986-994 (1996); Gibson, U. E. M, et al., Genome Research 6:995-1001 (1996); Holland, P. M., et al., Proc. Natl. Acad. Sci. USA 88:7276-7280, (1991); and Livak, K. J., et al., PCR Methods and Applications 357-362 (1995), each of which is incorporated by reference in its entirety), molecular beacons (Piatek, A. S., et al., Nat. Biotechnol. 16:359-63 (1998); Tyagi, S. and Kramer, F. R., Nature Biotechnology 14:303-308 (1996); and Tyagi, S. et al., Nat. Biotechnol. 16:49-53 (1998); herein incorporated by reference in their entireties), Invader assays (Third Wave Technologies, (Madison, Wis.)) (Neri, B. P., et al., Advances in Nucleic Acid and Protein Analysis 3826:117-125, 2000; herein incorporated by reference in its entirety), nucleic acid sequence-based amplification (NASBA; (See, e.g., Compton, J. Nucleic Acid Sequence-based Amplification, Nature 350: 91-91, 1991; herein incorporated by reference in its entirety), Scorpion probes (Thelwell, et al. Nucleic Acids Research, 28:3752-3761, 2000; herein incorporated by reference in its entirety), capacitive DNA detection (See, e.g., Sohn, et al. (2000) Proc. Natl. Acad. Sci. U.S.A. 97:10687-10690; herein incorporated by reference in its entirety), etc.

In some embodiments, the target nucleic acid sequences purified by the methods described herein are sequenced. Illustrative non-limiting examples of nucleic acid sequencing techniques include, but are not limited to, chain terminator (Sanger) sequencing and dye terminator sequencing, as well as “next generation” sequencing techniques. Those of ordinary skill in the art will recognize that because RNA is less stable in the cell and more prone to nuclease attack experimentally RNA is usually reverse transcribed to DNA before sequencing.

A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, DNA sequencing is achieved by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803; herein incorporated by reference in their entireties) the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. No. 6,787,308; U.S. Pat. No. 6,833,246; herein incorporated by reference in their entireties), the Lynx massively parallel signature sequencing technology (Brenner et al. (2000). Nat. Biotechnol. 18:630-634; U.S. Pat. No. 5,695,934; U.S. Pat. No. 5,714,330; herein incorporated by reference in their entireties) and the Adessi PCR colony technology (Adessi et al. (2000). Nucleic Acid Res. 28, E87; WO 00018957; herein incorporated by reference in its entirety).

In some embodiments, chain terminator sequencing is utilized. Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

A set of methods referred to as “next-generation sequencing” techniques have emerged as alternatives to Sanger and dye-terminator sequencing methods (Voelkerding et al., Clinical Chem., 55: 641-658, 2009; MacLean et al., Nature Rev. Microbiol., 7: 287-296; each herein incorporated by reference in their entirety). Next-generation sequencing (NGS) methods share the common feature of massively parallel, high-throughput strategies, with the goal of lower costs in comparison to older sequencing methods. NGS methods can be broadly divided into those that require template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. No. 6,210,891; U.S. Pat. No. 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 1×10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color and thus identity of each probe corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing in employed (see, e.g., Astier et al., J Am Chem Soc. 2006 Feb. 8; 128(5):1705-10, herein incorporated by reference). The theory behind nanopore sequencing has to do with what occurs when the nanopore is immersed in a conducting fluid and a potential (voltage) is applied across it: under these conditions a slight electric current due to conduction of ions through the nanopore can be observed, and the amount of current is exceedingly sensitive to the size of the nanopore. If DNA molecules pass (or part of the DNA molecule passes) through the nanopore, this can create a change in the magnitude of the current through the nanopore, thereby allowing the sequences of the DNA molecule to be determined.

In certain embodiments, HeliScope by Helicos BioSciences is employed (Voelkerding et al., Clinical Chem., 55: 641-658, 2009; MacLean et al., Nature Rev. Microbiol., 7: 287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

In certain embodiments, the Ion Torrent technology (Life Technologies) is employed to sequence purified target nucleic acid sequences. The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ˜99.6% for 50 base reads, with ˜100 Mb generated per run. The read-length is 100 base pairs. The accuracy for homopolymer repeats of 5 repeats in length is ˜98%. The benefits of ion semiconductor sequencing are rapid sequencing speed and low upfront and operating costs.

Another exemplary nucleic acid sequencing approach that may be adapted for use with the present invention was developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Patent Publication No. 20090035777.

Other emerging single molecule sequencing methods include real-time sequencing by synthesis using a VisiGen platform (Voelkerding et al., Clinical Chem., 55: 641-658, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

Another real-time single molecule sequencing system developed by Pacific Biosciences (Voelkerding et al., Clinical Chem., 55: 641-658, 2009; MacLean et al., Nature Rev. Microbiol., 7: 287-296; U.S. Pat. No. 7,170,050; U.S. Pat. No. 7,302,146; U.S. Pat. No. 7,313,308; U.S. Pat. No. 7,476,503; all of which are herein incorporated by reference) utilizes reaction wells 50-100 nm in diameter and encompassing a reaction volume of approximately 20 zeptoliters (10×10⁻²¹ L). Sequencing reactions are performed using immobilized template, modified phi29 DNA polymerase, and high local concentrations of fluorescently labeled dNTPs. High local concentrations and continuous reaction conditions allow incorporation events to be captured in real time by fluor signal detection using laser excitation, an optical waveguide, and a CCD camera.

In certain embodiments, the single molecule real time (SMRT) DNA sequencing methods using zero-mode waveguides (ZMWs) developed by Pacific Biosciences, or similar methods, are employed. With this technology, DNA sequencing is performed on SMRT chips, each containing thousands of zero-mode waveguides (ZMWs). A ZMW is a hole, tens of nanometers in diameter, fabricated in a 100 nm metal film deposited on a silicon dioxide substrate. Each ZMW becomes a nanophotonic visualization chamber providing a detection volume of just 20 zeptoliters (10-21 liters). At this volume, the activity of a single molecule can be detected amongst a background of thousands of labeled nucleotides.

The ZMW provides a window for watching DNA polymerase as it performs sequencing by synthesis. Within each chamber, a single DNA polymerase molecule is attached to the bottom surface such that it permanently resides within the detection volume. Phospholinked nucleotides, each type labeled with a different colored fluorophore, are then introduced into the reaction solution at high concentrations which promote enzyme speed, accuracy, and processivity. Due to the small size of the ZMW, even at these high, biologically relevant concentrations, the detection volume is occupied by nucleotides only a small fraction of the time. In addition, visits to the detection volume are fast, lasting only a few microseconds, due to the very small distance that diffusion has to carry the nucleotides. The result is a very low background.

Processes and systems for such real time sequencing that may be adapted for use with the invention are described in, for example, U.S. Pat. No. 7,405,281, entitled “Fluorescent nucleotide analogs and uses therefor;” U.S. Pat. No. 7,315,019, entitled “Arrays of optical confinements and uses thereof;’ U.S. Pat. No. 7,313,308, entitled “Optical analysis of molecules,” U.S. Pat. No. 7,302,146, entitled “Apparatus and method for analysis of molecules”, and U.S. Pat. No. 7,170,050, entitled “Apparatus and methods for optical analysis of molecules,” U.S. Patent Publications Nos. 20080212960, entitled “Methods and systems for simultaneous real-time monitoring of optical signals from multiple sources”, 20080206764, entitled “Flowcell system for single molecule detection”, 20080199932, entitled “Active surface coupled polymerases”, 20080199874, entitled “CONTROLLABLE STRAND SCISSION OF MINI CIRCLE DNA”, 20080176769, entitled “Articles having localized molecules disposed thereon and methods of producing same”, 20080176316, entitled “Mitigation of photodamage in analytical reactions”, 20080176241, entitled “Mitigation of photodamage in analytical reactions”, 20080165346, entitled “Methods and systems for simultaneous real-time monitoring of optical signals from multiple sources”, 20080160531, entitled “Uniform surfaces for hybrid material substrates and methods for making and using same”, 20080157005, entitled “Methods and systems for simultaneous real-time monitoring of optical signals from multiple sources”, 20080153100, entitled “Articles having localized molecules disposed thereon and methods of producing same”, 20080153095, entitled “CHARGE SWITCH NUCLEOTIDES”, 20080152281, entitled “Substrates, systems and methods for analyzing materials”, 20080152280, entitled “Substrates, systems and methods for analyzing materials”, 20080145278, entitled “Uniform surfaces for hybrid material substrates and methods for making and using same”, 20080128627, entitled “SUBSTRATES, SYSTEMS AND METHODS FOR ANALYZING MATERIALS”, 20080108082, entitled “Polymerase enzymes and reagents for enhanced nucleic acid sequencing”, 20080095488, entitled “SUBSTRATES FOR PERFORMING ANALYTICAL REACTIONS”, 20080080059, entitled “MODULAR OPTICAL COMPONENTS AND SYSTEMS INCORPORATING SAME”, 20080050747, entitled “Articles having localized molecules disposed thereon and methods of producing and using same”, 20080032301, entitled “Articles having localized molecules disposed thereon and methods of producing same”, 20080030628, entitled “Methods and systems for simultaneous real-time monitoring of optical signals from multiple sources”, 20080009007, entitled “CONTROLLED INITIATION OF PRIMER EXTENSION”, 20070238679, entitled “Articles having localized molecules disposed thereon and methods of producing same”, 20070231804, entitled “Methods, systems and compositions for monitoring enzyme activity and applications thereof’, 20070206187, entitled “Methods and systems for simultaneous real-time monitoring of optical signals from multiple sources”, 20070196846, entitled “Polymerases for nucleotide analogue incorporation”, 20070188750, entitled “Methods and systems for simultaneous real-time monitoring of optical signals from multiple sources”, 20070161017, entitled “MITIGATION OF PHOTODAMAGE IN ANALYTICAL REACTIONS”, 20070141598, entitled “Nucleotide Compositions and Uses Thereof’, 20070134128, entitled “Uniform surfaces for hybrid material substrate and methods for making and using same”, 20070128133, entitled “Mitigation of photodamage in analytical reactions”, 20070077564, entitled “Reactive surfaces, substrates and methods of producing same”, 20070072196, entitled “Fluorescent nucleotide analogs and uses therefore”, and 20070036511, entitled “Methods and systems for monitoring multiple optical signals from a single source”, and Korlach et aI. (2008) “Selective aluminum passivation for targeted immobilization of single DNA polymerase molecules in zero-mode waveguide nanostructures” Proc. Nat'I. Acad. Sci. U.S.A. 105(4): 11761181—all of which are herein incorporated by reference in their entireties.

The methods, compositions, systems, and devices of the present invention make use of samples which include, or are suspected of including, a target nucleic acid sequence. Samples may be derived from any suitable source, and for purposes related to any field, including but not limited to diagnostics, research, forensics, epidemiology, pathology, archaeology, etc. A sample may be biological, environmental, forensic, veterinary, clinical, etc. in origin. Samples may include nucleic acid derived from any suitable source, including eukaryotes, prokaryotes (e.g. infectious bacteria), mammals, humans, non-human primates, canines, felines, bovines, equines, porcines, mice, viruses, etc. Samples may contain, e.g., whole organisms, organs, tissues, cells, organelles (e.g., chloroplasts, mitochondria), synthetic nucleic acid, cell lysate, etc. Nucleic acid present in a sample (e.g. target nucleic acid, template nucleic acid, non-target nucleic acid, contaminant nucleic acid may be of any type, e.g., genomic DNA, RNA, plasmids, bacteriophages, synthetic origin, natural origin, and/or artificial sequences (non-naturally occurring), synthetically-produced but naturally occurring sequences, etc. Biological specimens may, for example, include FFPE, whole blood, lymphatic fluid, serum, plasma, sweat, tear, saliva, sputum, cerebrospinal (CSF) fluids, amniotic fluid, seminal fluid, vaginal excretions, serous fluid, synovial fluid, pericardial fluid, peritoneal fluid, pleural fluid, transudates, exudates, cystic fluid, bile, urine, gastric fluids, intestinal fluids, fecal samples, and swabs or washes (e.g., oral, nasopharangeal, optic, rectal, intestinal, vaginal, epidermal, etc.) and/or other biological specimens.

In some embodiments, samples that find use with the present invention are mixed samples (e.g. containing mixed nucleic acid populations). In some embodiments, samples analyzed by methods herein contain, or may contain, a plurality of different nucleic acid sequences. In some embodiments, a sample (e.g. mixed sample) contains one or more nucleic acid molecules (e.g., 1 . . . 10 . . . 10² . . . 10³ . . . 10⁴ . . . 10⁵ . . . 10⁶ . . . 10⁷, etc.) that contain a target sequence of interest in a particular application. In some embodiments, a sample (e.g. mixed sample) contains zero nucleic acid molecules that contain a target sequence of interest in a particular application. In some embodiments, a sample (e.g. mixed sample) contains nucleic acid molecules with a plurality of different sequences that all contain a target sequence of interest. In some embodiments, a sample (e.g. mixed sample) contains one or more nucleic acid molecules (e.g. 1 . . . 10 . . . 10² . . . 10³ . . . 10⁴ . . . 10⁵ . . . 10⁶ . . . 10⁷, etc.) that do not contain a target sequence of interest in a particular application. In some embodiments, a sample (e.g. mixed sample) contains zero nucleic acid molecules that do not contain a target sequence of interest in a particular application. In some embodiments, a sample (e.g. mixed sample) contains nucleic acid molecules with a plurality of different sequences that do not contain a target sequence of interest. In some embodiments, a sample contains more nucleic acid molecules that do not contain a target sequence than nucleic acid molecules that do contain a target sequence (e.g. 1.01:1 . . . 2:1 . . . 5:1 . . . 10:1 . . . 20:1 . . . 50:1 . . . 10²:1 . . . 10³:1 . . . 10⁴:1 . . . 10⁵:1 . . . 10⁶:1 . . . 10⁷:1). In some embodiments, a sample contains more nucleic acid molecules that do contain a target sequence than nucleic acid molecules that do not contain a target sequence (e.g. 1.01:1 . . . 2:1 . . . 5:1 . . . 10:1 . . . 20:1 . . . 50:1 . . . 10²:1 . . . 10³:1 . . . 10⁴:1 . . . 10⁵:1 . . . 10⁶:1 . . . 10⁷:1). In some embodiments, a sample contains a single target sequence which may be present in one or more nucleic acid molecules in the sample. In some embodiments, a sample contains a two or more target sequences (e.g. 2, 3, 4, 5 . . . 10 . . . 20 . . . 50 . . . 100, etc.) which may each be present in one or more nucleic acid molecules in the sample.

In some embodiments, various sample processing steps may be accomplished to prepare the nucleic acid molecules within a sample, including, but not limited to cell lysis, restriction digestion, purification, precipitation, resuspension (e.g. in amplification buffer), dialysis, etc. In some embodiments, sample processing is performed before or after any of the steps of the present invention including, but not limited to amplification, amplicon detection, amplicon isolation, sequencing, etc.

### EXAMPLES

### Example 1

### Exemplary Enrichment Protocol

This Example describes an exemplary protocol that can be employed for target nucleic acid sequence purification.

### Hybridization

A) Using sterile aerosol barrier pipettor tip, prepare hybridization solution by mixing equal volume of mLysis_{DNA} buffer (Abbott) and water.
B) In a 1.5 mL screw cap tube, mix 300 ul of hybridization solution with 250 ng to 1 ug of genomic DNA extracted from FFPE samples, 16.5 ul of Human Cot1 DNA or other suitable blocker DNA (final concentration: 0.05 mg/ml) and 2 Capture Baits per each target designed based on the targets of interest (one from each complementary strand, final concentration: 20 pmol each). The final hybridization mixture is 330 ul. Exemplary capture sequences that could be used for BRAF, kRAS, CASC1, and cKit are shown in Table 1 below:

TABLE 1

| | Length | |
|---|---|---|
| | of target | |
| | specific | |
| | sequences + | |
| | Length of | |
| | poly A | |
| Bait Name | stretches | Sequences (5′-3′) |
| BRAF bait | 28 + 10 | 5′-Biotin-TEG-AAA AAA AAA |
| #1 | | ACT GTT TTC CTT TAC TTA |
| | | CTA CAC CTC AG-3Phos |
| | | (SEQ ID NO: 1) |
| BRAF bait | 43 + 10 | 5′-Biotin-TEG-AAA AAA AAA |
| #2 | | AGAC CTT CAA TGA CTT TCT |
| | | AGT AAC TCA GCA GCA TCT |
| | | CAG GGC C-3Phos |
| | | (SEQ ID NO: 2) |
| kRas-bait1F | 32 + 10 | 5′Biotin-C6 AAAAAAAAAAGAAA |
| | | ACTGTAACAATAAGAGTGGAGATAGC |
| | | TG-3Phos |
| | | (SEQ ID NO: 3) |
| kRas-bait2R | 30 + 10 | 5′Biotin-C6-AAAAAAAAAATCAA |
| | | AGAATGGTCCTGCACCAGTAATATG |
| | | C-3Phos |
| | | (SEQ ID NO: 4) |
| CASC1- | 25 + 10 | 5′Biotin-C6-AAAAAAAAAAGGTG |
| bait1 | | AAGCAGAATCAGTGGTCGCCC- |
| | | 3Phos |
| | | (SEQ ID NO: 5) |
| cKit-exon8- | 30 + 10 | 5′Biotin-C6-AAAAAAAAAAAGGA |
| bait1F | | TTCCCAGAGCCCACAATAGATTGGT |
| | | A-3Phos |
| | | (SEQ ID NO: 6) |
| cKit-exon8- | 32 + 10 | 5′Biotin-C6-AAAAAAAAAATAAT |
| bait2R | | CATCTCACCTCTGCTCAGTTCCTGGA |
| | | CA-3Phos |
| | | (SEQ ID NO: 7) |

Note:
TEG stands for tetra-ethyleneglycol, a 15 atom spacer. C6 is a 6-carbon linker between the biotin molecule and nucleotides. 3Phos stands for 3' Phosphate to block the extension by DNA polymerase. TEG and C6 serve as a linker between baits and beads to eliminate the stereo hindrance for target hybridization. 6 carbon linkers also have been compared with TEG linker, showing no significant difference in term of capturing efficiency. Multiple target-specific baits may be pooled together with DNA samples in single tubes.

C) Mix the hybridization mixtures by vortex.
D) Incubate the tubes at 92 C for 10 minutes.
E) After incubation, remove and place the tubes on ice for 1 minute followed by incubation at room temperature for 60 minutes on a rocker.

### Microparticle Capture

A) Re-suspend microparticles by vortexing until particles are in suspension and settled particles are no longer seen on the bottom of the bottle.
B) First estimate the total amount of microparticles needed for the total number of capture reactions (n). Add 5×n uL (5 ul, equivalent to 50 ug microparticles per capture) of microparticle into a new 1.5 mL tube on a nonmagnetic rack, add 1 ml of hybridization solution at room temperature.
C) Place the tubes in a magnetic capture stand for 1 minute to allow the particles to be captured on the side of the tubes.
D) With the tubes in the magnetic capture stand, use a clean sterile pipettor tip to carefully remove the liquid from each tube and discard the fluid into a liquid waste container. Remove the fluid as completely as possible. Use a new tip for every sample.
E) Remove the tubes from the magnetic rack and transfer to a non-magnetic rack. Re-suspend the microparticles in 20×n ul of hybridization solution.
F) Transfer 20 ul of the microparticles to each hybridization reaction tube, and vortex the mixtures until a uniform suspension is obtained. Incubate at room temperature for 20 minutes.
G) After the incubation is complete, place the 1.5 mL tubes in a magnetic capture stand for 1-2 minutes to allow the particles to be captured on the side of the tube.
H) With the tubes in the magnetic capture stand, use a clean 1000 uL sterile pipettor tip to carefully remove the liquid from each tube and discard the fluid into a liquid waste container. Remove the fluid as completely as possible. Do not disturb or aspirate the captured magnetic particles. Use a new tip for every sample.
I) Remove the tube from the magnetic rack and transfer to a non-magnetic rack.

### Washing of Capture DNA

A) Using a clean 1000 uL pipettor tip for each sample, add 1000 uL of 0.1×SSC buffer pre-warmed to 40 C to the samples and re-suspend the magnetic particles by vortexing. Incubate for 10 minutes at 40 C.
B) Place the tubes in a magnetic capture stand for 1-2 minutes to allow the particles to be captured on the side of the tubes.
C) Using a clean 1000 uL pipettor tip, remove the washing solution from the tubes and discard fluid into a liquid waste container. Use a new tip for every sample.
D) Remove the tubes from the magnetic rack and transfer to a non-magnetic rack.
E) Repeat washing two additional times using 1000 uL of 0.×SSC for each wash.

### Elution of Captured DNA

A) Using a clean 1000 uL pipettor tip for each sample, add 300 uL of water to the samples and re-suspend the magnetic particles by vortexing.
B) Incubate the tubes at 92 C for 5 minutes.
C) Remove the tubes from the heating block and place in a magnetic capture stand for one minute to allow the particles to be captured on the side of the tubes.
D) Using a clean 1000 uL pipettor tip for each sample, transfer the Eluted Sample to a clean 1.5 mL screw top microfuge tube. Do not disturb or aspirate the captured microparticles.

### Example 2

### Capture Efficiency Assessment

This Example describes experiments conducted to determine capture efficiency using the protocol in Example 1 and the BRAF and cKit capture sequences in Table 1 above. Capture specificity was evaluated using target sequences located on chromosomes different from the targets of interest as an indicator for non-specific capture. The relative enrichment efficiency can be calculated as the ratio between capture efficiency of the target sequences and that of the non-specific background sequences. Real-time PCR was used in lieu of NextGen sequencing as a method to estimate the capture efficiency, which is calculated based on the Ct values for each locus with and without enrichment steps.

Table 2 below shows efficiency of a thyroid FFPE DNA using baits specific to either BRAF or c-Kit or both.

TABLE 2

| | Specific Capture | | Non-specific Capture |
|---|---|---|---|
| | BRAF | c-KIT | β-globin |
| Bait | (ch*7) | (ch4) | (ch11) |
| BRAF | 21.24% | 0.00% | 0.00% |
| c-KIT | 0.00% | 17.92% | 0.01% |
| BRAF&c-KIT | 15.82% | 10.88% | 0.01% |

*Ch = choromosome

Table 3 below shows the capture efficiency of a melanoma FFPE DNA using baits specific to either BRAF or c-Kit or both.

TABLE 3

| | Specific Capture | | Non-specific Capture |
|---|---|---|---|
| | BRAF | c-KIT | β-globin |
| Bait | (ch7) | (ch4) | (ch11) |
| BRAF | 17.80% | 0.00% | 0.00% |
| c-KIT | 0.02% | 33.80% | 0.00% |
| BRAF&c-KIT | 18.69% | 22.14% | 0.02% |

Table 4 below shows the correlation between the capture efficiency and the distance between bait and targeted region at the BRAF locus.

TABLE 4
Distance from Bait

| Sample | <100 bp | >20 kbp | ∞ (ch4) | ∞ (ch11) | ∞ (ch12) |
|---|---|---|---|---|---|
| Thyroid cancer | 21.24% | 0.00% | 0.00% | 0.00% | 0.00% |
| Melanoma | 17.80% | 0.04% | 0.00% | 0.00% | 0.00% |
| Cell FFPE 1 | 37.37% | 0.05% | 0.03% | 0.01% | 0.00% |
| Cell FFPE 2 | 26.33% | 0.10% | 0.13% | 0.04% | 0.20% |

Although the invention has been described in connection with specific preferred embodiments, it should be understood that the invention as claimed should not be unduly limited to such specific embodiments. Various modification and variation of the described methods and compositions of the invention will be apparent to those skilled in the art without departing from the scope and spirit of the invention. Indeed, various modifications of the described modes for carrying out the invention understood by those skilled in the relevant fields are intended to be within the scope of the following claims. All publications and patents mentioned in the present application are herein incorporated by reference.

We claim:

1. A method of separating target nucleic acid sequences from a target sample comprising:
a) contacting a target sample with bait molecules to generate a mixed sample,
wherein said target sample comprises a population of target and non-target nucleic acid sequences, and
wherein said bait molecules: i) are free in solution in said mixed sample, and ii) each comprises a ligand and a target capture sequence which is 18 to 48 bases in length;
b) heating said mixed sample to a nucleic acid denaturation temperature;
c) incubating said mixed sample at a hybridization temperature such that said target capture sequences hybridize to said target nucleic acid sequences, wherein said incubating is conducted for no more than 4 hours before performing steps d), e) and f);
d) adding magnetic binding particles to said mixed sample, wherein said magnetic binding particles comprise ligand binding moieties;
e) incubating said mixed sample under conditions such that said bait molecules bind to said magnetic binding particles via said ligands binding to said ligand binding moieties thereby generating a population of target nucleic acid sequence linked magnetic particles; and
f) magnetically separating said target nucleic acid sequence linked magnetic binding particles from said mixed sample thereby generating a population of separated target nucleic acid sequence linked magnetic binding particles.

2. The method of claim 1, wherein said incubating in step c) is conducted for no more than 1.5 hours before performing steps d), e) and f).

3. The method of claim 1, wherein said target capture sequence is 22-35 bases in length.

4. The method of claim 1, wherein said mixed sample, during said incubating in step c), has, or is treated to have, a salt concentration of at least 1.3 M.

5. The method of claim 1, wherein said hybridization temperature in step c) is about 15-30 degrees Celsius.

6. The method of claim 1, wherein said hybridization temperature in step c) is about room temperature.

7. The method of claim 1, further comprising washing said population of separated target nucleic acid sequence linked magnetic particles with a wash solution.

8. The method of claim 7, wherein said washing is conducted at a temperature of about 30-50 degrees Celsius.

9. The method of claim 1, wherein, in step a), said target sample and said bait molecules are further contacted with carrier nucleic acid in order to generate said mixed sample.

10. The method of claim 9, wherein said carrier nucleic acid comprises blocking oligonucleotides and/or human repetitive nucleic acid sequences.

11. The method of claim 1, further comprising contacting said population of separated target nucleic acid sequence linked magnetic binding particles with an aqueous solution at a denaturation temperature, and eluting said target nucleic acid sequences away from said magnetic binding particles to generate a population of eluted target nucleic acid sequences.

12. The method of claim 1, wherein the total amount of nucleic acid in said target sample is between 100 nanograms and 5.0 micrograms.

13. The method of claim 1, wherein said target nucleic acid sequences are pathogenic nucleic acid sequences and said non-target nucleic acid sequences are human nucleic acid sequences.

14. A method of separating target nucleic acid sequences from a target sample comprising:
a) contacting a target sample with bait molecules to generate a mixed sample,
wherein said target sample comprises a population of target and non-target nucleic acid sequences, and
wherein said bait molecules: i) are free in solution in said mixed sample, and ii) each comprises a ligand and a target capture sequence which is 23 to 39 bases in length;
b) heating said mixed sample to a nucleic acid denaturation temperature;
c) incubating said mixed sample at about room temperature such that said target capture sequences hybridize to said target nucleic acid sequences, wherein said incubating is conducted for no more than 2 hours before performing steps d), e) and f);
d) adding magnetic binding particles to said mixed sample, wherein said magnetic binding particles comprise ligand binding moieties;
e) incubating said mixed sample under conditions such that said bait molecules bind to said magnetic binding particles via said ligands binding to said ligand binding moieties thereby generating a population of target nucleic acid sequence linked magnetic particles; and
f) magnetically separating said target nucleic acid sequence linked magnetic binding particles from said mixed sample thereby generating a population of separated target nucleic acid sequence linked magnetic binding particles.

15. The method of claim 14, wherein said mixed sample, during said incubating in step c), has, or is treated to have, a salt concentration of at least 1.3 M.

16. A composition or system comprising:
a) a solution comprising bait molecules, wherein said bait molecules are free in solution, and wherein said bait molecules comprise a ligand and a target capture sequence 18 to 48 bases in length;
b) magnetic binding particles, wherein said magnetic binding particles comprise ligand binding moieties.

17. The composition or system of claim 16, wherein said target capture sequence is 25-35 bases in length.

18. The composition or system of claim 16, wherein said ligand comprises biotin and said ligand binding moieties comprise streptavidin.

19. The composition of claim 16, wherein said solution has a salt concentration of at least 1.3 M.

20. The composition of claim 16, wherein said solution has a salt concentration of at least 1.9 M.
